(19) United States
(12) Patent Application Publication
(10) Pub. No.: US 2012/0208264 A1
(43) Pub. Date: Aug. 16, 2012

(54) METHOD AND APPARATUS FOR DETECTION OF LIVIGN PHYTOPLANKTON CELLS IN WATER

(75) Inventors: KROON BERND, Jeddah (SA); Anja Kornmueller, Bremen (DE)

(73) Assignee: RWO GmbH

(21) Appl. No.: 13/455,325

(22) Filed: Apr. 25, 2012

Related U.S. Application Data
(62) Division of application No. 12/439,676, filed on Aug. 10, 2009.

(30) Foreign Application Priority Data
Sep. 1, 2006 (DE) 10 2006 041 347.4

(51) Int. Cl. C12M 1/34

(52) U.S. Cl. 435/288.7

(57) ABSTRACT

The invention relates to a method and an apparatus for detecting living phytoplankton cells and/or microorganisms in or out of water, particularly ballast water, bodies of water, sewage, or water in swimming and bathing devices. Said method is characterized by the following steps: the variable fluorescence (Fv) is calculated by forming the difference between the maximum fluorescence (Fm) and the minimum fluorescence (Fo) in a measuring space or detecting part or all of the dynamic shape of a fluorescence induction curve in a measuring space, particularly measuring; and calculating the number of living phytoplankton cells and/or microorganisms of a reference species in the measuring space in accordance with the variable fluorescence (Fv).

### CROSS-REFERENCE TO RELATED APPLICATIONS

The present application is a Divisional Application U.S. patent application Ser. No. 12/439,676, filed Aug. 10, 2009, which is a U.S. National Stage Application under 35 U.S.C. §371 of PCT Application Serial No. PCT/EP2007/006812, filed Aug. 1, 2007, which in turn claims the priority benefit of German Patent Application No. 10 2006 041 347.4, filed Sep. 1, 2006, the entireties of which are incorporated herein by reference.

### BACKGROUND OF THE INVENTION

The invention relates to a method and apparatus for detection of viable, i.e., living, phytoplankton cells and/or microorganisms in/from water, in particular surface water such as ponds, rivers, streams, lakes and dammed-up rivers, fresh water and brackish water, ballast water of ships, deep sea water, ground water and trickle water, process water, industrial water, cooling water and circulating water, wastewater, bath water and swimming pool water, culturing water and culture media or production water.

### SUMMARY OF THE INVENTION

In the use of natural water, the removal of phytoplankton and/or microorganisms is an important goal in the processing of water to make the water usable for various purposes and to maintain processing limits and discharge limits. For example, one familiar problem involves the development of phytoplankton mass in surface water and the breakthrough in the filter systems and occurrence in drinking water distribution networks. When processing is inadequate, problems occur due to the phytoplankton itself, such as discoloration of the water, odors and toxins as well as the multiplication of other unwanted bacteria in the water, which in turn use phytoplankton as a nutrient source. At the same time, exposure and/or entrainment of phytoplankton species in species-alien biotopes is undesirable because this causes a shift in the ecological equilibrium.

It is therefore necessary to detect living phytoplankton cells in water, in particular as part of monitoring and control methods online, especially for controlling a treatment method for removal and/or disinfection of the living phytoplankton cells and/or microorganisms in water.

However, the methods known today are unable to reliably yield results within reasonable time limits, in particular for cell sizes less than or equal to 0.1 mm and with the required cell count determination, in particular in the presence of different species and with any composition. Thus, although the known method of biomass reproduction is very sensitive, it is also very time consuming because it takes days or even weeks to determine the biomass. Another disadvantage of this method is that the original cell count remains unknown. Therefore, this method is not suitable for online monitoring.

In addition, the passive fluorometric method with which the biomass of the phytoplankton cells can be determined in a water sample is also known. One disadvantage of this method is that it does not provide any information about living or dead phytoplankton cells because no differentiation is possible.

The arrangement of a delivery pump for delivering the water is advantageous.

A control unit by means of which the analyzer unit and/or one or more valves and/or a delivery pump and/or a removal and/or disinfection device can be controlled is preferably provided. This allows verifiable documentation.

### BRIEF DESCRIPTION OF THE FIGURES

The invention will now be explained in greater detail on the basis of the figures, in which,

FIG. 1 shows an example of a fluorescence induction curve;

FIG. 2 shows a diagram of an exemplary embodiment of the apparatus for detection of living phytoplankton cells and/or microorganisms in water;

FIG. 3 shows the relationship between the variable fluorescence Fv and the number of living cells per milliliter in a measurement volume.

### DETAILED DESCRIPTION OF THE INVENTION

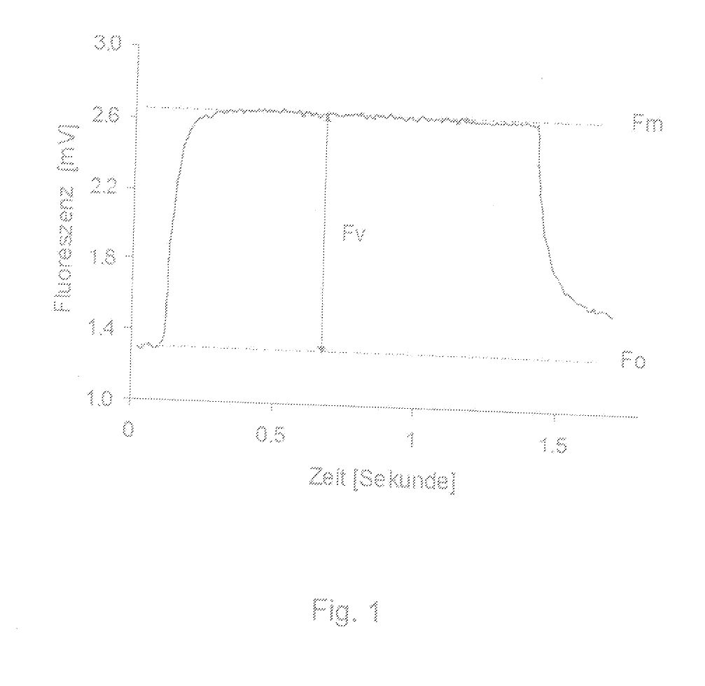

FIG. 1 shows a measured fluorescence induction curve 1 plotted over time. The level of minimal fluorescence Fo is reached on induction of a state in which practically all primary electron acceptors are still oxidized according to a state in darkness or by using a light source of a light with a wavelength of more than 700 nm. By activating an intermittent or continuous light source at a point in time T1, photochemical reactions are activated, resulting in the primary electron acceptors being reduced. When at least approximately all the primary electron acceptors are reduced, the fluorescence level reaches the maximal fluorescence Fm. The fluorescence induction curve 1 over time after turning on the light source at point in time T1 reveals that an increase in fluorescence from the minimal fluorescence Fo to the maximal fluorescence Fm does not take place suddenly, but instead the increase is continuous in a dynamic process, which is to be attributed to the behavior of the living phytoplankton cells.

The decline in the fluorescence induction curve 1 after turning off the light source at point in time T2 is also shown in FIG. 1.

The variable fluorescence Fv which corresponds to the difference in the maximal fluorescence Fm minus the minimal fluorescence Fo is proportional to the number of living cells within the measurement space 11. On the basis of a relationship between variable fluorescence Fv and the number of living phytoplankton cells and/or microorganisms of a certain predefined reference species found as part of a calibration procedure, it is thus possible to calculate the number of living phytoplankton cells and/or microorganisms of the reference species as a function of the variable fluorescence Fv.

As an alternative to calculating the number of living phytoplankton cells on the basis of the variable fluorescence Fv as the difference between the maximal fluorescence Fm and the minimal fluorescence Fo, it is possible to determine the number of living phytoplankton cells and/or microorganisms from the dynamic characteristic of the fluorescence induction curve 1 with the help of a mathematical model.

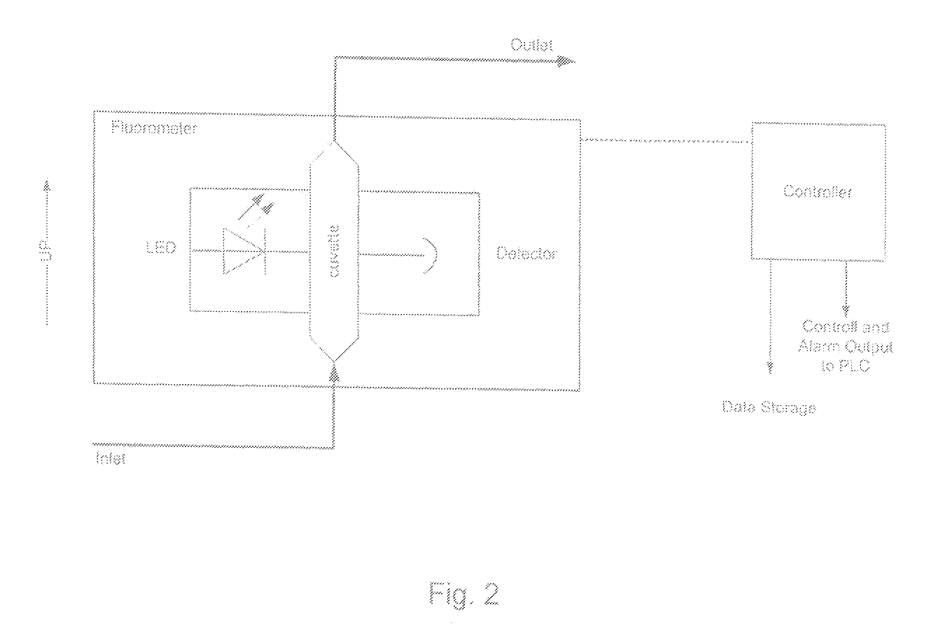

FIG. 2 shows a diagram of an exemplary embodiment of an apparatus for detection of living phytoplankton cells and/or microorganisms in/from water.

The apparatus has a fluorometer 10 as well as an analysis, monitoring and control unit 20.

The test space 11 of the fluorometer 10 is formed by a cuvette with two parallel glass surfaces 12 and 13.

The fluorometer 10 has light sources 14 in the form of three types of LEDs, one light source emitting light of a wavelength of more than 700 nm, a pulsating and a continuous light source. In addition, the fluorometer 10 has a detector 15. The light source 14 is controlled, i.e., turned on and off, by the control unit 20. By means of the detector 15 it is possible to detect the fluorescence induction curve of the fluorescence of the amount of water present in the cuvette and to be tested, i.e., in particular the minimal fluorescence Fo when the light source 14 is turned off and the maximal fluorescence Fm when the light source 14 is turned on and to transmit this information to the control unit 20 for further analysis.

The fluorometer 10 is tied into a continuous or discontinuous delivery process and has an inlet 16 and an outlet 17.

By means of the control unit 20, there is an analysis of the measured values of the fluorescence transmitted over a data line 18 from the fluorometer 10 by calculating the variable fluorescence Fv by forming the difference between the maximal fluorescence Fm and the minimal fluorescence Fo and then calculating the number of living phytoplankton cells and/or microorganisms of a reference species in the water in the cuvette to be tested from this calculated variable fluorescence by means of a previously measured and stored calibration line.

In addition, control unit 20 monitors the results for when a predefined and stored limit value is exceeded, such that when the limit value is exceeded an alarm is delivered over the data line 21.

The control unit 20 is connected via a data line 22 to a memory unit, by means of which the number of living phytoplankton cells and/or microorganisms to be calculated can be stored.

This apparatus is tied into a continuous delivery stream and thus allows continuous online monitoring of the water to be tested.

The monitoring device is connected upstream and/or downstream from a removal and/or disinfection unit (not shown here) which receives its control commands from the control unit 20.

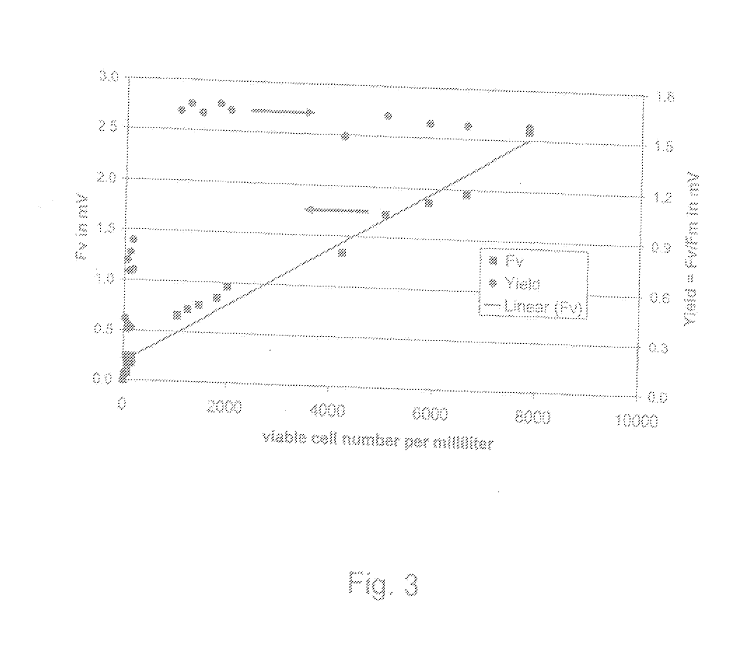

FIG. 3 shows the relationship between the variable fluorescence Fv and the number of living cells per milliliter in a measurement volume.

The International Maritime Organization gives limit values for certain size classes of microorganisms as the discharge standard for ballast water treatment systems on board ships.

The size range of ≧10 μm to <50 μm is dominated by phytoplankton. A discharge limit of <10 living microorganisms in the smallest length per milliliter is stipulated for this standard.

So far, only monitoring by tedious microscopic counting on land has been possible. However, online monitoring in real time is necessary because the ballast water is discharged directly into the environment.

In this example according to FIG. 3, the marine green algae Tetraselmis suecica with a size of 10 μm was used as the reference microorganism for the method.

FIG. 3 shows the Fv signal as a function of the microscopically counted living Tetraselmis cell count in the inflow and outflow of a ballast water treatment system consisting of a mechanical preseparation followed by a disinfection treatment.

As FIG. 3 shows, the qualitative yield, which is generally used (corresponding to Fv/Fm=(Fm−Fo)/Fm) does not have any correlation with the living cell count.

However, the Fv signal determined by the inventive method and used further yields a definite linear dependence on the living cell count (R2=0.98) even over a very wide range of cell counts.

The conversion of this signal for living biomass to live cell count is based on a volumetric relationship.

Equivalent cell counts for cell sizes other than the reference species can also be calculated based on the third power root. The slope of the proportionality line is a measure of the size of the individual cells. This relationship is shown in the following table:

| | Slope of Tetraselmis/ | |
|---|---|---|
| | slope of species | |
| Species | Slope ratio | X^{(1/3)} |
| Thalassiosira weissflogii | 1.34 | 1.10 |
| Isochrysis galbana | 7.27 | 1.94 |
| Nannochloropsis oculata | 73.80 | 4.19 |

The advantage of the third power root dependence is that living phytoplankton cells less than 10 μm in size hardly have any effect on the signal at all even in larger numbers whereas living phytoplankton cells larger than 10 μm cause a significant signal even in a lower cell count of less than 10 per milliliter. Reliable monitoring of this limit value can therefore be ensured.

The so-called active fluorometric method serves to determine the quantum efficiency of the photosynthesis system, which can be specific only for living cells. One disadvantage of this method is that it does not allow quantification of phytoplankton cells.

The object of the invention is to create a method and an apparatus for detection of living phytoplankton cells and/or microorganisms in/from water, making it possible to determine the number of living phytoplankton cells and/or microorganisms in a water sample with little effort and in the shortest possible amount of time and in particular to allow online monitoring of water.

This object is achieved by a method according to claim 1 or claim 2 and an apparatus according to claim 19.

The inventive method for detection of living phytoplankton cells and/or microorganisms in or from water comprises the following steps:
Calculating the variable fluorescence Fv by forming the difference between the minimal fluorescence Fo and the maximal fluorescence Fm in a measurement space, regardless of its geometry, containing the water to be tested and/or the microorganisms to be tested, or by determining the dynamic characteristic of a fluorescence induction curve in a measurement space, regardless of its geometry, containing the water and/or microorganisms to be tested, in particular a measurement of the characteristic of the fluorescence induction curve over time and calculation of the maximal fluorescence Fm by calculating the time integral of the fluorescence induction curve or by interpolation of the fluorescence induction curve, and
Calculating the number of living phytoplankton cells and/or microorganisms of a reference species in the measurement space as a function of the variable fluorescence Fv.

Alternatively, the inventive method according to claim 2 comprises the following steps:
Measuring the heat evolved in a measurement space (11) due to an input of light and
Calculating the number of living phytoplankton cells and/or microorganisms of one reference species in the measurement space (11) as a function of the heat evolved.

The minimal fluorescence Fo refers to the fluorescence from both living and dead cells, while the maximal fluorescence Fm corresponds to the fluorescence at which at least approximately all primary electron acceptors have been reduced and the variable fluorescence Fv corresponds to the difference between the maximal fluorescence Fm and the minimal fluorescence Fo, each based on the water and/or microorganisms to be tested in the measurement space.

To determine the biological material in water, the fluorescence may be detected by a fluorometer. Two states can be differentiated, first the minimal fluorescence Fo (dark state) and the maximal fluorescence Fm with input of light, in particular light of a predetermined wavelength. It has surprisingly been found that the difference between the maximal fluorescence Fm and the minimal fluorescence Fo, i.e., the variable fluorescence Fv, is a measure of the number of living phytoplankton cells and/or microorganisms in the measurement space and/or the test quantity of water and/or microorganisms because the variable fluorescence Fv and the number of living fluorescence cells show a correlation.

The number of living phytoplankton cells and/or microorganisms of one reference species in the measurement space and/or the test quantity of water and/or microorganisms can be calculated by measuring the minimal fluorescence Fo (without illumination), measuring the maximal fluorescence Fm (with illumination) and calculating the variable fluorescence Fv by forming the difference Fm minus Fo.

With the inventive apparatus for detection of living phytoplankton cells and/or microorganisms in/from water, at least one fluorometer is provided for determining the minimal fluorescence Fo and the maximal fluorescence Fm of a quantity of water and/or a quantity of microorganisms within a test space, such that the fluorometer has at least one light source and at least one detector. In addition, an analyzer unit is provided, by means of which the variable fluorescence Fv is determined and the number of living phytoplankton cells and/or microorganisms of a reference species in the test volume may be calculated as a function of the variable fluorescence Fv thereby determined.

Alternatively or in addition to the calculation of variable fluorescence Fv by forming the difference between the maximal fluorescence Fm and the minimal fluorescence Fo, it is also possible with the inventive method and the inventive apparatus to determine the dynamic characteristic of a fluorescence induction curve in a measurement space, in particular by partial or complete determination of the characteristic of the fluorescence induction curve over time, and obtaining the missing information by interpolation using a mathematical model.

The intensity of the fluorescent light is directly proportional to the number of cells of a reference species in the measurement space and/or the test quantity in/from the water, i.e., the relationship follows a straight line, where the slope of the proportionality line is in turn a measure of the size of the individual cells.

The “test space” may be a test volume which is filled with the water to be tested, i.e., a water sample, but it may also be a membrane filter through which a certain quantity of water to be tested has been filtered, and whereby the minimal fluorescence Fo and the maximal fluorescence Fm are measured with the cell layer on the surface of the membrane filter without water.

It has surprisingly been found that it is possible to determine the number of living cells on the basis of the calculation of the variable fluorescence Fv as well as on the basis of the measurement of the heat evolved by the living cells due to an input of light, i.e., a short-term light exposure of the cells in a measurement space.

The variable fluorescence Fv and the heat evolved are thus equally a measure of the number of living cells in the measurement volume, i.e., these two variables are equivalent to one another in particular inasmuch as living cells evolve both heat and fluorescence after a brief exposure to light and thus the number of living cells of a reference species in a measurement space can be calculated as a function of the variable fluorescence as well as alternatively or additionally as a function of the heat evolved.

Other advantageous embodiments of the invention are defined in the dependent claims.

It is thus advantageous if a linear calibration is performed to determine the relationship between the variable fluorescence Fv and the number of living phytoplankton cells and/or microorganisms of a reference species in the measurement space, in particular a reference species of a cell size of more than 0.8 μm in the smallest length it is advantageous in particular that the linear calibration is performed once or several times before the measurement for determination of the fluorescence Fo, Fm.

The reference species and the cell size of more than 0.8 μm in the smallest length can be determined and/or ascertained by known microscopic methods, i.e., the reference species is preselectable.

On the basis of this linear calibration, the cell count can now be calculated from the value of the variable fluorescence Fv thereby determined.

There is preferably a calculation of an equivalent number of living phytoplankton cells and/or microorganisms of cell sizes other than the reference species, in particular by volumetric comparison. The cells contents of phytoplankton cells are usually proportional to the volume of the cells. On the basis of this correlation, an equivalent number of living phytoplankton cells and/or microorganisms can be calculated when the cell sizes are different from the cell size of the reference species.

There is preferably a determination of the scattered light, in particular before the determination of the minimal fluorescence Fo and/or the maximal fluorescence Fm. The scattered light can be determined directly before performing the measurement, in particular 50 μs to 100 μs, especially 80 μs before determination of the minimal fluorescence Fo and/or the total fluorescence Fm. This measurement detects any scattered light that may be present or any other form of light not emitted by living cells.

The minimal fluorescence Fo is preferably determined by forming the average of several individual measurements of the minimal fluorescence within the test volume. Preferably several individual measurements are performed in intervals of 20 ms to 100 ms.

Alternatively or additionally, the maximal fluorescence Fm may be determined by forming an average from several individual measurements of the maximal fluorescence Fm; in particular several individual measurements may be performed at intervals of 20 ms to 100 ms.

By forming an average of minimal fluorescence Fo and/or maximal fluorescence Fm, the precision of the measurement can be increased. Since several individual measurements can be performed in a very short chronological order, this does not result in any relevant time lag in application of the method, in particular as part of an online monitoring of an apparatus for monitoring water quality.

It is advantageous if the variable fluorescence Fv is calculated by using an average of the minimal fluorescence Fo and/or an average of the maximal fluorescence Fm. The quality. and accuracy can also be increased in this way.

The determination of the minimal fluorescence Fo and the maximal fluorescence Fm is preferably performed by means of a fluorometer using at least one pulsating light source PL and/or at least continuous light source KL, whereby LEDs in particular are used as the light sources PL and KL.

The minimal fluorescence Fo is preferably determined by using pulsating light, in particular light at a wavelength of 420 nm.

Reaching the state for determination of the minimal fluorescence Fo can preferably be accelerated by using a light source with a wavelength longer than 700 run, using in particular LEOs as the light source.

The minimal fluorescence Fo is preferably determined by using at least one pulsating light source PL, in particular light pulses of the pulsating light source PL with an interval of 20 ms to 100 ms.

The maximal fluorescence Fm is preferably determined by using continuous light. in particular with a wavelength of 660 nm.

It is advantageous if the fluorescence is determined by using at least one pulsating light source PL, in particular light pulses of the pulsating light source PL with an interval of 20 ms to 100 ms and at least one continuous light source KL.

As part of a continuous monitoring, the method, i.e., the individual steps of the method, may be repeated in a predefined number. This makes it possible to perform continuous monitoring and quality assurance of the water to be tested and to implement it as part of online monitoring. In particular, an alarm may be triggered automatically by monitoring for a predefined limit value.

It is advantageous if, in a continuous monitoring process, test volumes or test quantities are removed repeatedly from a supply of water and/or a stream of water and if the steps of the process are each applied one or more times to each test volume and the respective calculated number of living phytoplankton cells and/or microorganisms is transmitted to a monitoring and/or control unit. In particular the data thereby ascertained, i.e., the calculated number of living phytoplankton cells and/or microorganisms in the measurement space may be sent to a device with which the removal of cells from the water and/or disinfection of the water is/are controlled and/or monitored.

A treatment method for removing and/or disinfecting the living phytoplankton cells and/or microorganisms in/from the water may thus be controlled as a function of the calculated number of living phytoplankton cells and/or microorganisms and/or as a function of a calculated equivalent number of living phytoplankton cells and/or microorganisms.

Volatile or permanent storage of at least the calculated number of living phytoplankton cells and/or microorganisms, in particular for documentation purposes, is preferred.

It is advantageous if the calculated number of living phytoplankton cells and/or microorganisms in the water is monitored for exceeding a predefinable limit value in particular triggering an alarm when the limit value is exceeded.

The inventive apparatus for detection of living phytoplankton and/or microorganisms in/from water preferably has a measurement space which is formed by a cuvette in particular, especially a cuvette made of glass or plastic.

The test space preferably has an inlet and/or an outlet. The test space may in particular be embedded in a continuous or cycled delivery stream of the water to be tested, i.e., integrated into a delivery line via the inlet and the outlet.

The apparatus preferably has at least one pulsating light source and/or at least one continuous light source, LEDs in particular being used as the light source.

Multiple light sources are preferably arranged, in particular at least one light of pulsating light, especially blue light with a wavelength of approx. 420 nm and in particular at least one light source of continuous light, in particular red light with a wavelength of approx. 660 nm and in particular a light source with a wavelength of longer than 700 nm.

A device for removing cells from water and/or for disinfection of water is preferably connected upstream and/or downstream from the apparatus.

Preferably at least one controllable valve is provided on an inlet and/or outlet of the test space. This makes it possible to tie the apparatus into a continuous delivery process, such that the delivery of the water to be tested can be interrupted by means of the controllable valve for the period of time of a measurement by means of the fluorometer.

1-28. (canceled)

29. An apparatus for detection of living phytoplankton cells and/or microorganisms in a water source having at least one fluormeter for determination of minimal and maximal fluorescence within a test space wherein the fluormeter has at least one light source and at least one detector and further wherein an analyzer unit is provided for determination of the variable fluorescence and whereby the number of living phytoplankton cells and/or microorganisms of a reference species in at test space can be calculated as a function of the variable fluorescence.

30. The apparatus according to claim 29 characterized in that the test space is formed by a cuvette, in particular made of glass or plastic.

31. The apparatus according to claim 29 wherein the test has at least one port to allow the entry of removal of water.

32. The apparatus according to claim 29 wherein at least one pulsating light source and/or at least one continuous light source provided.

33. The apparatus according to claim 29 wherein a plurality of light sources are provided and further wherein at least one light source is selected from the group of pulsating light sources having a wavelength of approximately 420 nm, and continuous light sources having wavelengths of approximately 660 nm to greater than 700 nm.

34. The apparatus according to claim 29 further including a removal and/or disinfection device in fluid communication with the apparatus.

35. The apparatus according to claim 29 further including at least one controllable value in fluid communication with the test space.

36. The apparatus according to claim 29 further including a delivery pump for delivering water.

37. The apparatus according to claim 30 further including a control unit for controlling the analyzer and further including at least one valve, a delivery pump and a removal and/or disinfection device in fluid communication with the test space.

38. The apparatus according to claim 29 further including a memory unit for storing at least one measurement selected from the group comprising fluorescence values, the variable fluorescence and the calculated number of living phytoplankton cells and/or microorganisms.
